(19) United States
(12) Patent Application Publication
(10) Pub. No.: US 2022/0309527 A1
(43) Pub. Date: Sep. 29, 2022

(54) Systems and Methods for Promoting Patient Health

(71) Applicant: BayCare Health System, Inc., Clearwater, FL (US)

(72) Inventors: Edward Rafalski, Clearwater, FL (US); Brian Curtiss, Clearwater, FL (US); Ashish Jain, Clearwater, FL (US); Kelli Graf, St. Petersburg, FL (US); John Beauvais, Clearwater, FL (US)

(73) Assignee: BayCare Health System, Inc., Clearwater, FL (US)

(21) Appl. No.: 17/214,204

(22) Filed: Mar. 26, 2021

(51) Int. Cl. G06Q 30/02; G16H 40/20; G06Q 10/10

(52) U.S. Cl. CPC G06Q 30/0236; G16H 40/20; G06Q 10/1095; G06Q 30/0226

(57) ABSTRACT

Systems and methods for promoting patient health and customer loyalty to healthcare providers through a healthcare loyalty program include a registration process for allowing a user to create a user account by submitting user information. The system includes a database in which customer information related to existing customers of a provider is stored, a user validation process for confirming that the user is an existing customer of the provider by comparison of the user information with customer information stored in the database, a scheduling process for the user to schedule an appointment for a service provided by the provider, and a gift card process for generating an instruction to generate and deliver the gift card to the user based on the command received from the scheduling process. The system sends a command to generate and deliver a gift card after registration is completed and the user account is created.

### FIELD OF THE INVENTION

The invention relates to healthcare. More particularly, the invention relates to systems and methods for promoting patient health and customer loyalty to healthcare providers through a healthcare loyalty program.

### BACKGROUND

Loyalty programs have been used in many industries to induce customers to continuing purchasing certain goods and services from the same seller or produced by the same manufacturer. Often such programs have taken the form of rewards points programs in which a customer is rewarded for purchases by being given points that can be redeemed to reduce the purchase price of the same or similar goods or services purchased in the future from the same seller or manufacturer. In other cases, discounts are given to encourage the customer to continue buying the same product or service or other products or services from the same seller or manufacturer. Such programs are effective in maintaining brand loyalty among consumers as well as loyalty to particular retailers or manufacturers. Such loyalty programs are not known to be used by healthcare providers nor are they used by merchants or service providers of any type to encourage a customer to make an appointment using an online scheduling process. Further, such loyalty programs have not been used to promote patient health by encouraging the making of appointments with a healthcare service provider.

A need exists for systems and methods that promote patient health and customer loyalty to healthcare providers through a healthcare loyalty program. A further need exists for systems and methods that promote patient health and customer loyalty to healthcare providers by providing gift cards or other items of value after registration of a patient for an appointment to receive healthcare services from a particular healthcare provider. Still a further need exists for encouraging a customer seeking healthcare services to make an appointment with a healthcare service provider by making the scheduling of the appointment easier using an online scheduling process.

### SUMMARY

The invention relates to systems and methods for promoting patient health and customer loyalty to healthcare providers through a healthcare loyalty program. The systems and methods include a registration process for allowing a user to create a user account by submitting user information. The system also includes a database in which customer information related to existing customers of a provider is stored and a user validation process for confirming that the user is an existing customer of the provider by comparison of the user information with customer information stored in the database. The system further includes a scheduling process for the user to schedule an appointment for a service provided by the provider. A gift card process of the system generates an instruction to generate and deliver a gift card to the user based on a command received from the system. The system sends a command to generate and deliver a gift card after registration is completed and the user account is created.

The systems and methods described herein provide advantages and benefits by promoting patient health and customer loyalty to healthcare providers through a healthcare loyalty program. The systems and methods described herein also provide advantages and benefits by promoting patient health and customer loyalty to healthcare providers by providing gift cards or other items of value after registration of a patient for an appointment to receive healthcare services from a particular healthcare provider. The systems and methods described herein also provide advantages and benefits by encouraging a customer seeking healthcare services to make an appointment with a healthcare service provider using an online scheduling process.

Accordingly, the invention features a system for incentivizing a user to schedule a service with a provider by providing a reward after accessing a platform for making an appointment. The system includes a computing device having a processor and associated memory, a display screen for displaying information and images produced or received by the computing device, and a telecommunications network. The system also includes a registration process for allowing a user to create a user account by submitting user information. The registration process is accessible by the user via the telecommunications network using the computing device. The system further includes a database in which customer information related to existing customers of a provider is stored for access and retrieval, a user validation process for confirming that the user is an existing customer of the provider by comparison of the user information with customer information stored in the database, a scheduling process for the user to schedule an appointment for a service provided by the provider, and a gift card process for generating an instruction to generate and deliver a gift card to the user based on the command received from the system. The system sends a command to generate and deliver a gift card after registration is completed and the user account is created.

In another aspect, the invention can feature the command being generated by the registration process or by the scheduling process.

In another aspect, the invention can feature the command being or including data related to the amount of the gift card, redemption information, or both.

In another aspect, the invention can feature the redemption information of the gift card being or including a bar code, an alphanumeric code, or both.

In another aspect, the invention can feature the command further being or including information related to the user, the user information, the user account, or a combination of two or more of the foregoing.

In another aspect, the invention can feature the user validation process generating a validated user determination if the user is determined to be an existing customer and a non-user determination if the user is determined not to be an existing customer.

In another aspect, the invention can feature the command being generated and sent when or after the user account is created by the registration process.

In another aspect, the invention can feature the command being generated and sent when or after the user schedules the appointment with the provider using the scheduling process.

In another aspect, the invention can feature the gift card being or including a physical gift card, an electronic gift card, or both.

In another aspect, the invention can feature the scheduling process analyzing the customer information in the database and making an affirmative determination if the customer information includes at least one record of an appointment being scheduled and completed by the user and making a negative determination if the customer information does not include at least one record of an appointment being scheduled and completed by the user.

In another aspect, the invention can feature the scheduling process sending the command to the gift card process only if the appointment is a first appointment by the user with the provider.

In another aspect, the invention can feature the scheduling process accessing a database to determine if the appointment is a first appointment with the provider or a subsequent appointment, wherein the database includes records of each appointment scheduled and attended by the user.

In another aspect, the invention can feature the records of each appointment include one or more scheduled appointment records and one or more attended appointment records that correspond to the one or more scheduled appointment records. Each of the one or more scheduled appointment records has a corresponding attended appointment record. Each of the one or more attended appointment records includes an “attended” record if the user attended the scheduled appointment and a “not attended” record if the user did not attend the scheduled appointment.

In another aspect, the invention can feature the scheduling process sending the command to the gift card process only if the appointment is a first appointment by the user with the provider.

In another aspect, the invention can feature the scheduling process sending the command to the gift card process if the appointment is a first appointment or a subsequent appointment by the user with the provider.

The invention also features a system for incentivizing a user to schedule a service with a provider by providing a reward after accessing a platform for making an appointment. The system includes a computing device having a processor and associated memory, a display screen for displaying information and images produced or received by the computing device, and a telecommunications network. The system also includes a registration process for allowing a user to create a user account by submitting user information. The registration process is accessible by the user via the telecommunications network using the computing device. The system includes a database in which customer information related to existing customers of a provider is stored for access and retrieval via the telecommunications network using the computing device. The system also includes a user validation process for confirming that the user is an existing customer of the provider by comparison of user information associated with the user with customer information stored in the database. The system further includes a scheduling process for the user to schedule an appointment for a service provided by the provider. The system also includes a reward process for generating an instruction to generate and deliver a reward to the user based on the command received from the registration process or the scheduling process. The reward is or includes a physical or electronic item of value.

In another aspect, the invention can feature the reward being or including a gift card. The gift card can be or include a physical gift card, an electronic gift card, or both.

A method of the invention can be used for incentivizing a user to schedule a service with a provider by providing a reward after accessing a platform for making an appointment. The method includes the step of: (a) providing a system that includes: (i) a computing device having a processor and associated memory; (ii) a display screen for displaying information and images produced or received by the computing device; (iii) a telecommunications network; (iv) a registration process for allowing a user to create a user account by submitting user information, wherein the registration process is accessible by the user via the telecommunications network using the computing device; (v) a database in which customer information related to existing customers of a provider is stored for access and retrieval via the telecommunications network using the computing device; (vi) a user validation process for confirming that the user is an existing customer of the provider by comparison of user information associated with the user with customer information stored in the database; (vii) a scheduling process for the user to schedule an appointment for a service provided by the provider; and (viii) a reward process for generating an instruction to generate and deliver a reward to the user based on the command received from the registration process or the scheduling process. The method also includes the steps of: (b) creating the user's user account using the registration process to submit the user's user information; (c) confirming that a user is an existing customer of the provider by using the user validation process to compare user information associated with the user with customer information stored in the database; (d) scheduling an appointment for the user using the scheduling process for a service provided by the provider; (e) sending a command to the reward process to generate and deliver a reward; and (f) generating an instruction via the reward process to generate and deliver a reward to the user based on the command, wherein the reward is or includes a physical or electronic item of value.

Another method of the invention can include the reward being or including a gift card. The gift card can be or include a physical gift card, an electronic gift card, or both.

Another method of the invention can include the step of: (g) analyzing the customer information in the database and making an affirmative determination if the customer information includes at least one record of an appointment being scheduled and completed by the user and making a negative determination if the customer information does not include at least one record of an appointment being scheduled and completed by the user.

Another method of the invention can include the step of: (h) sending the command to the reward process only if the appointment is a first appointment by the user with the provider.

Another method of the invention can include the step of: (i) sending the command to the reward process if the appointment is a first appointment or a subsequent appointment by the user with the provider.

Another method of the invention can include the step of: (j) sending the command to the reward process only if the appointment is a subsequent appointment by the user with the provider.

Another method of the invention can include the step of: (k) accessing a database using the scheduling process and determining if the appointment is a first appointment with the provider or a subsequent appointment, wherein the database includes records of each appointment scheduled and attended by the user.

Another method of the invention can include the records of each appointment including one or more scheduled appointment records and one or more attended appointment records that correspond to the one or more scheduled appointment records. Each of the one or more scheduled appointment records includes a corresponding attended appointment record. Each of the one or more attended appointment records includes an “attended” record if the user attended the scheduled appointment and a “not attended” record if the user did not attend the scheduled appointment.

Unless otherwise defined, all technical terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents and other references mentioned herein are incorporated by reference in their entirety. In the case of conflict, the present specification, including definitions will control.

### BRIEF DESCRIPTION OF THE DRAWINGS

FIG. 1 is a flow chart of a new user registration process of a system for promoting patient health.

FIG. 2 is a flow chart of a registered user sign-in process of the system of FIG. 1.

FIG. 3 is a flow chart of a user validation process of the system of FIG. 1.

FIG. 4 is a flow chart of a gift card process of the system of FIG. 1.

FIG. 5 is a process for retrieving a forgotten user name or resetting a password of the system of FIG. 1.

FIG. 6 is a screen shot of a registration page of a system for promoting patient health.

FIG. 7 is a screen shot of a sign-in page of the system of FIG. 1.

FIG. 8 is a screen shot of a password reset page of the system of FIG. 1.

FIG. 9 is a screen shot of an account activation page of the system of FIG. 1.

FIG. 10 is a screen shot of a welcome page of the system of FIG. 1.

FIG. 11 is a screen shot of a benefits page of the system of FIG. 1 before an account has been upgraded to receive additional benefits and a reward.

FIG. 12 is a screen shot of an account upgrade page of the system of FIG. 1.

FIG. 13 is a screen shot of the benefits page of the system of FIG. 1 after an account has been upgraded to receive additional benefits and a reward.

FIG. 14 is a screen shot of a “my providers” page of the system of FIG. 1, which provides the user with information concerning the user's provider.

FIG. 15 is a screen shot of an account settings page of the system of FIG. 1.

### DETAILED DESCRIPTION

The present invention is best understood by reference to the detailed drawings and description set forth herein. Embodiments of the invention are discussed below with reference to the drawings; however, those skilled in the art will readily appreciate that the detailed description given herein with respect to these figures is for explanatory purposes as the invention extends beyond these limited embodiments. For example, in light of the teachings of the present invention, those skilled in the art will recognize a multiplicity of alternate and suitable approaches, depending upon the needs of the particular application, to implement the functionality of any given detail described herein beyond the particular implementation choices in the following embodiments described and shown. That is, numerous modifications and variations of the invention may exist that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

The present invention should not be limited to the particular methodology, compounds, materials, manufacturing techniques, uses, and applications, described herein, as these may vary. The terminology used herein is used for the purpose of describing particular embodiments only, and is not intended to limit the scope of the present invention. As used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” may be a reference to one or more steps or means and may include sub-steps and subservient means.

All conjunctions used herein are to be understood in the most inclusive sense possible. Thus, a group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should be read as “and/or” unless expressly stated otherwise. Structures described herein are to be understood also to refer to functional equivalents of such structures. Language that may be construed to express approximation should be so understood unless the context clearly dictates otherwise.

Unless otherwise defined, all terms (including technical and scientific terms) are to be given their ordinary and customary meaning to a person of ordinary skill in the art, and are not to be limited to a special or customized meaning unless expressly so defined herein.

Terms and phrases used in this application, and variations thereof, especially in the appended claims, unless otherwise expressly stated, should be construed as open ended as opposed to limiting. As examples of the foregoing, the term “including” should be read to mean “including, without limitation,” “including but not limited to,” or the like; the term “having” should be interpreted as “having at least”; the term “includes” should be interpreted as “includes but is not limited to”; the term “example” is used to provide exemplary instances of the item in discussion, not an exhaustive or limiting list thereof; and use of terms like “preferably,” “preferred,” “desired,” “desirable,” or “exemplary” and words of similar meaning should not be understood as implying that certain features are critical, essential, or even important to the structure or function of the invention, but instead as merely intended to highlight alternative or additional features that may or may not be utilized in a particular embodiment of the invention.

Those skilled in the art will also understand that if a specific number of an introduced claim recitation is intended, such an intent will be explicitly recited in the claim, and in the absence of such recitation no such intent is present. For example, as an aid to understanding, the appended claims may contain usage of the introductory phrases “at least one” and “one or more” to introduce claim recitations; however, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.).

All numbers expressing dimensions, quantities of ingredients, reaction conditions, and so forth used in the specification are to be understood as being modified in all instances by the term “about” unless expressly stated otherwise. Accordingly, unless indicated to the contrary, the numerical parameters set forth herein are approximations that may vary depending upon the desired properties sought to be obtained.

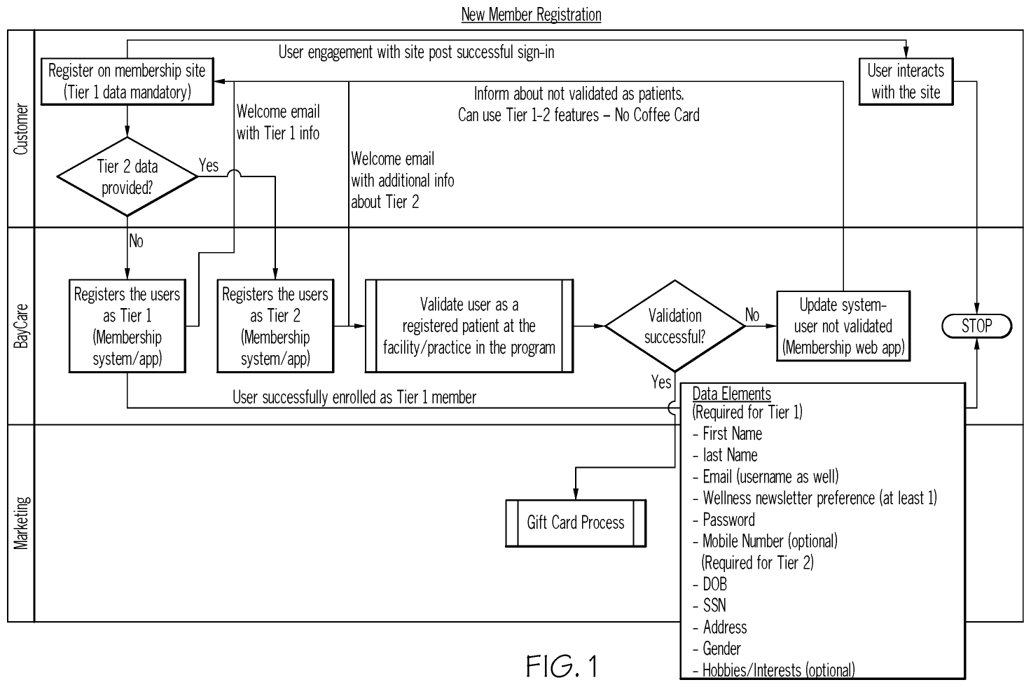

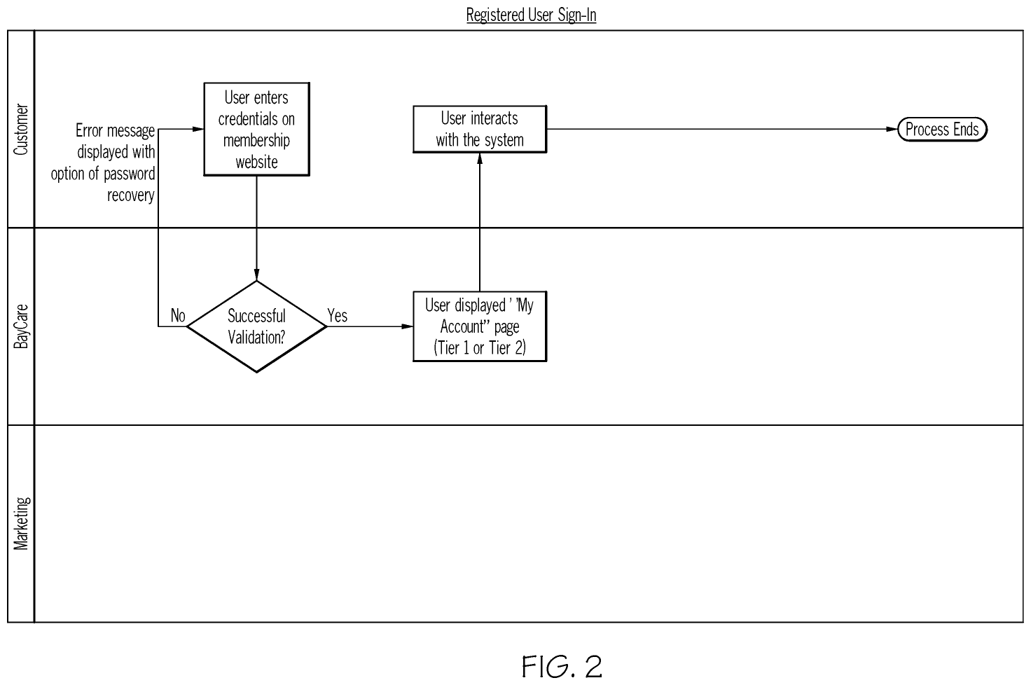

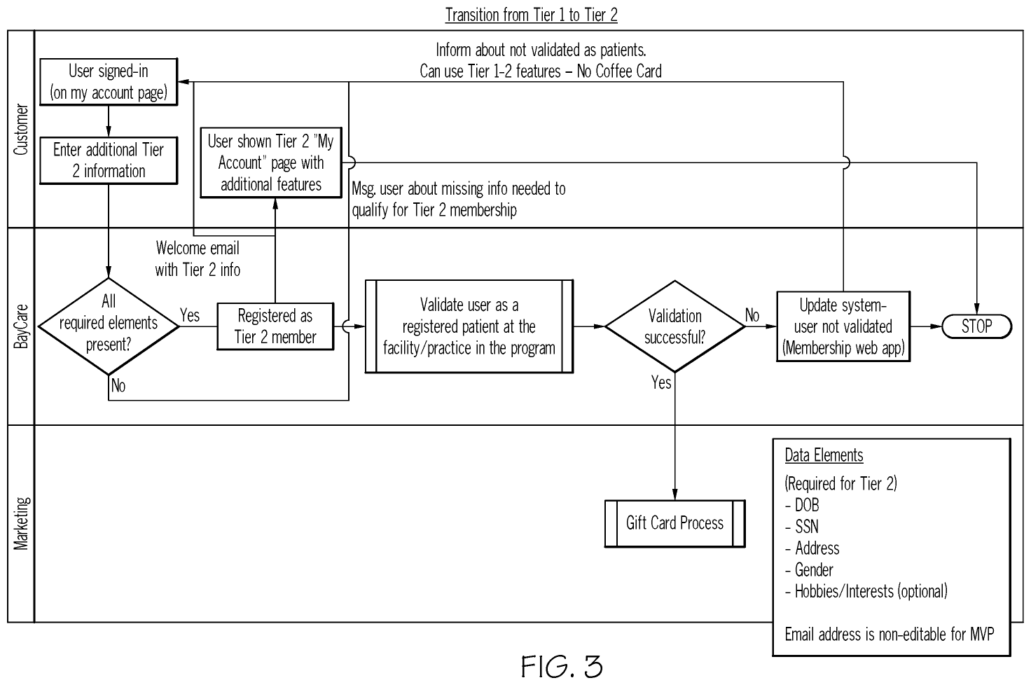

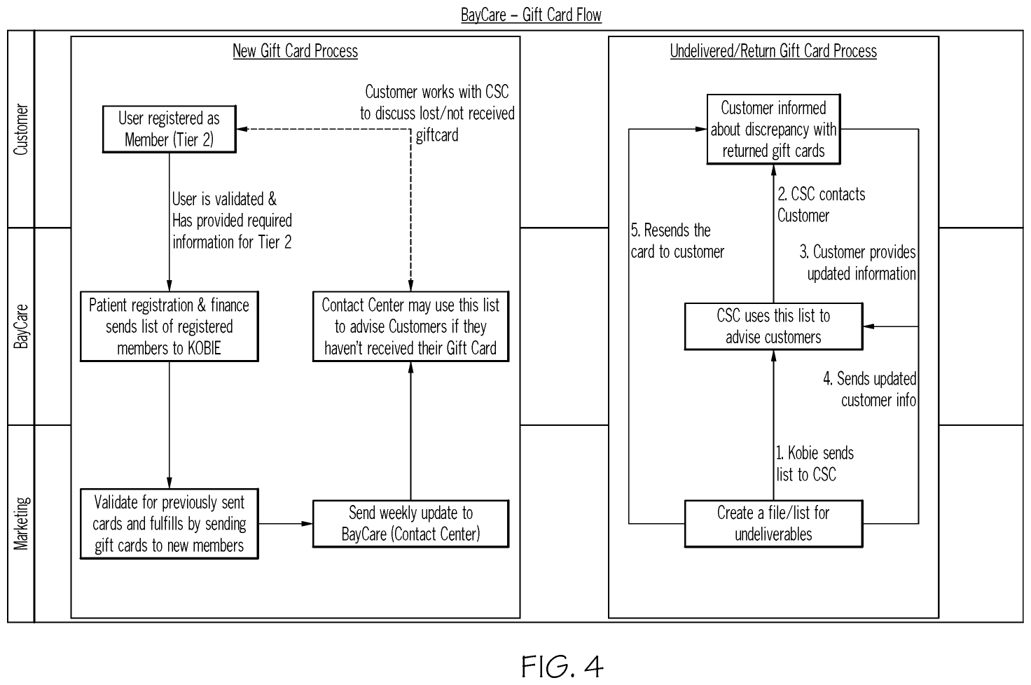

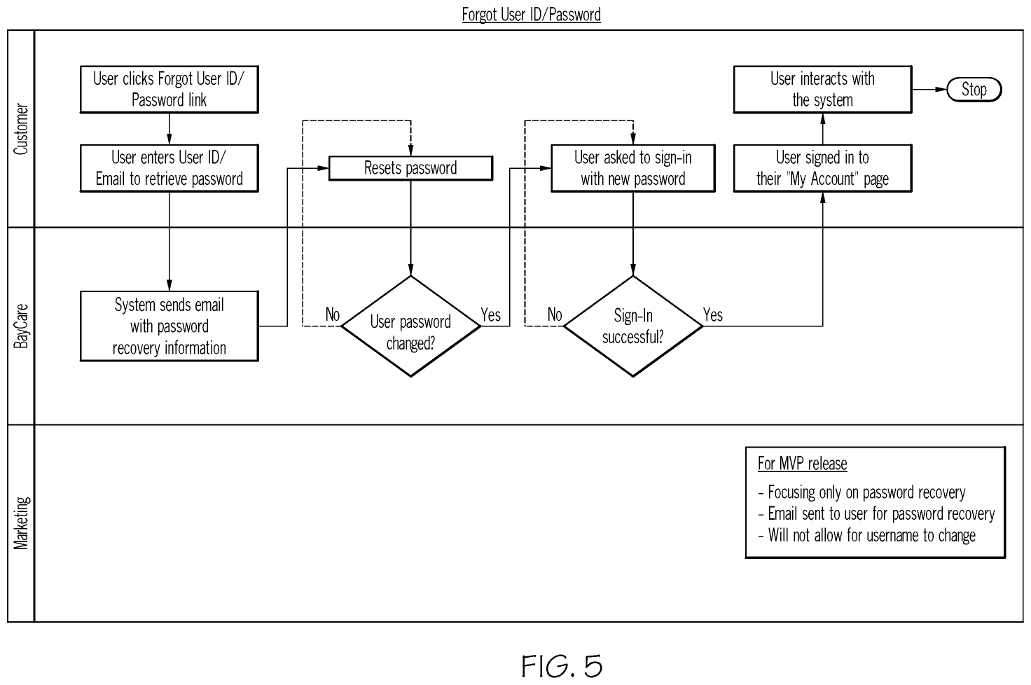

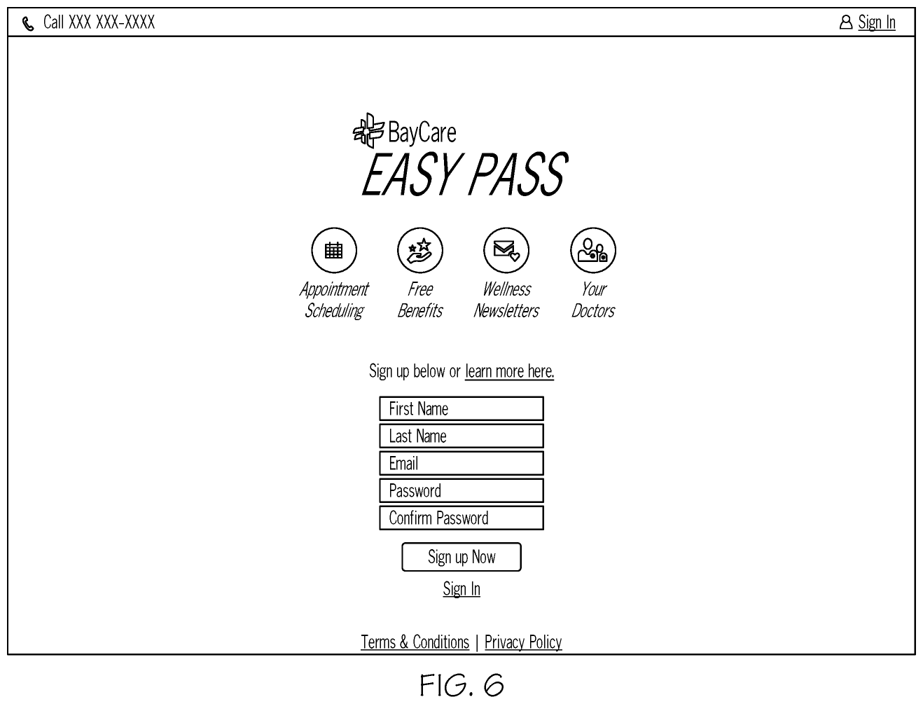

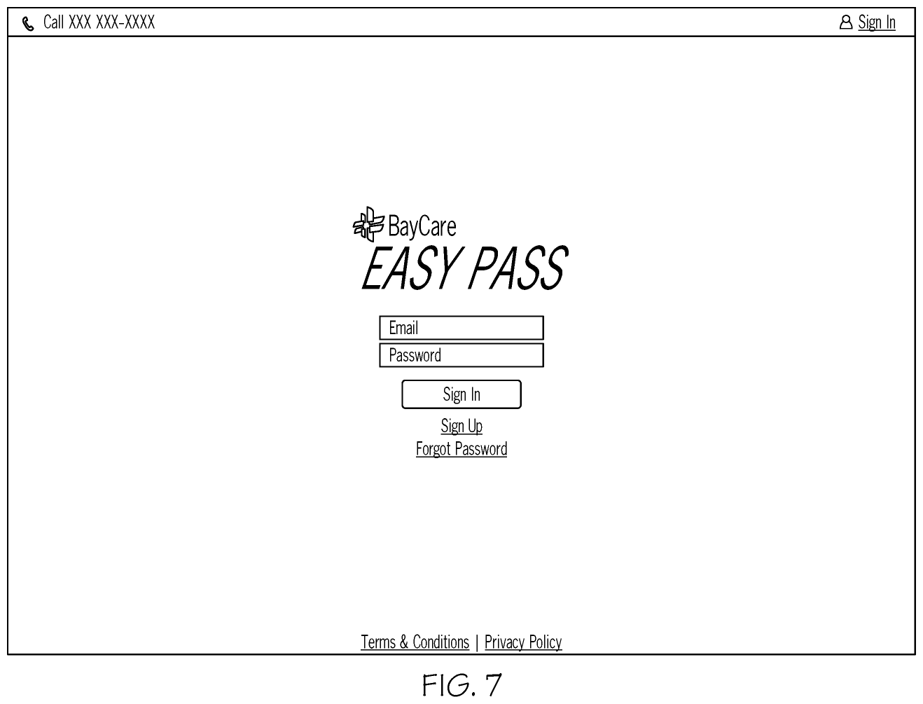

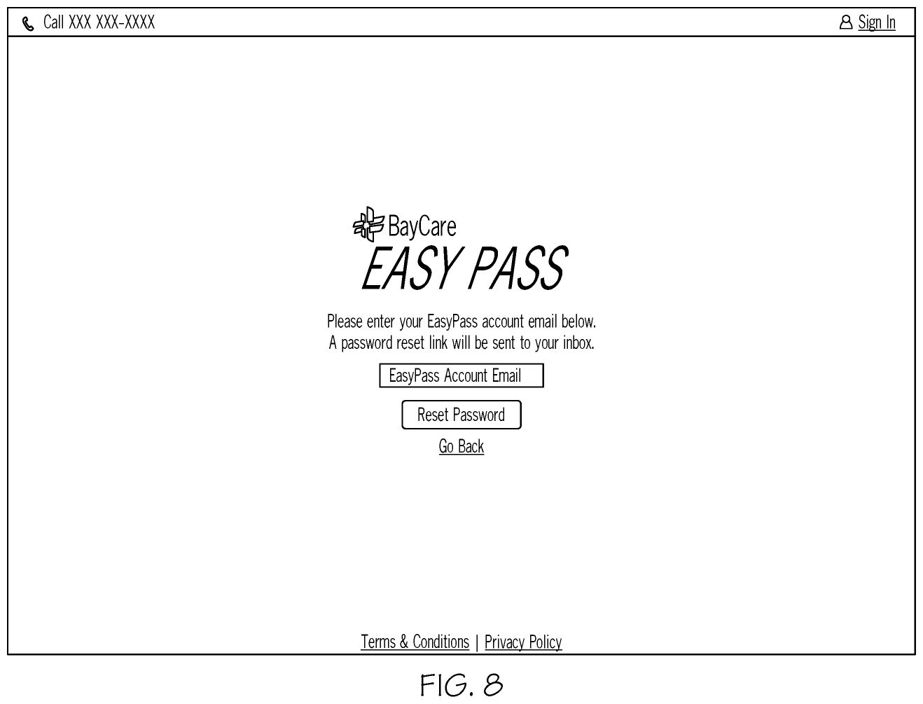

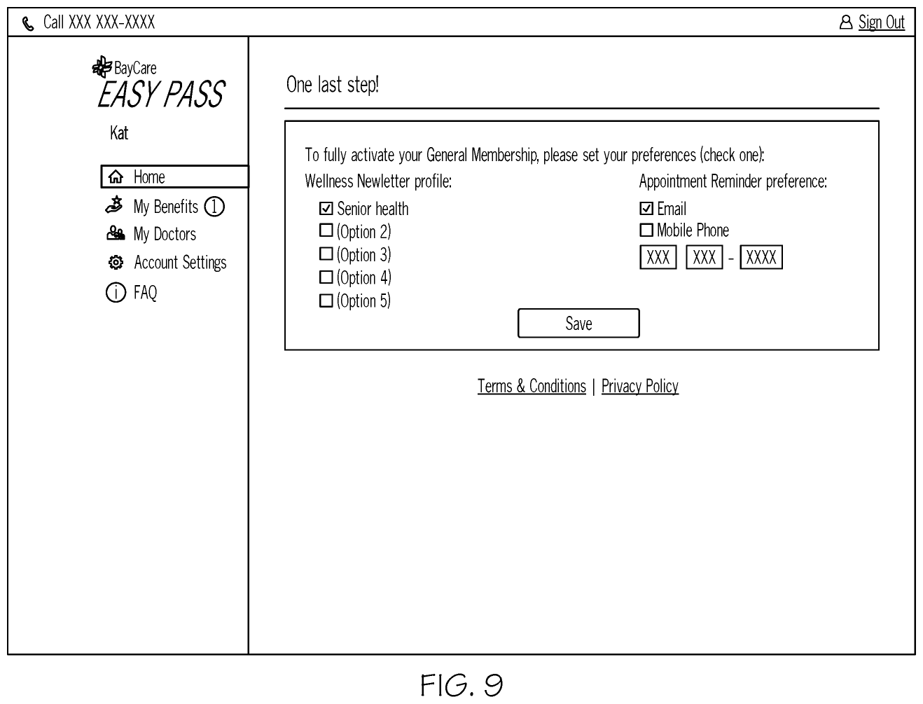

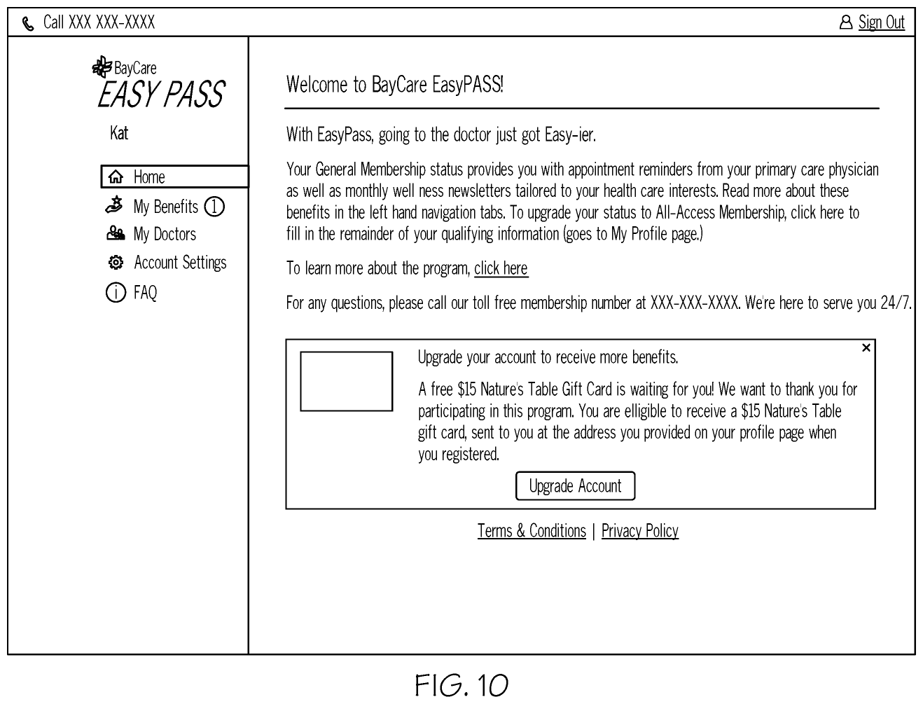

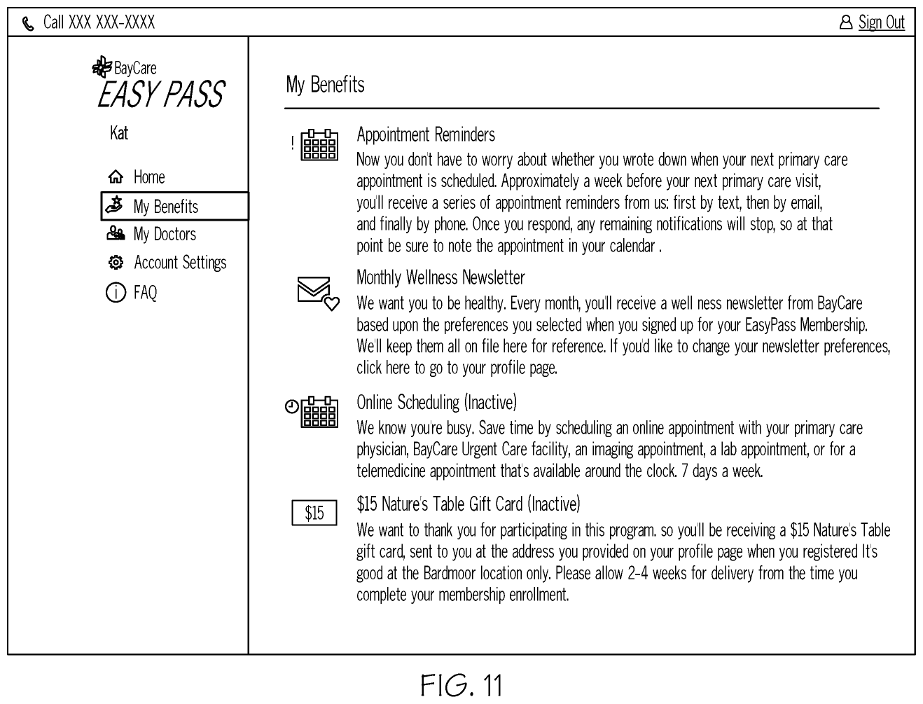

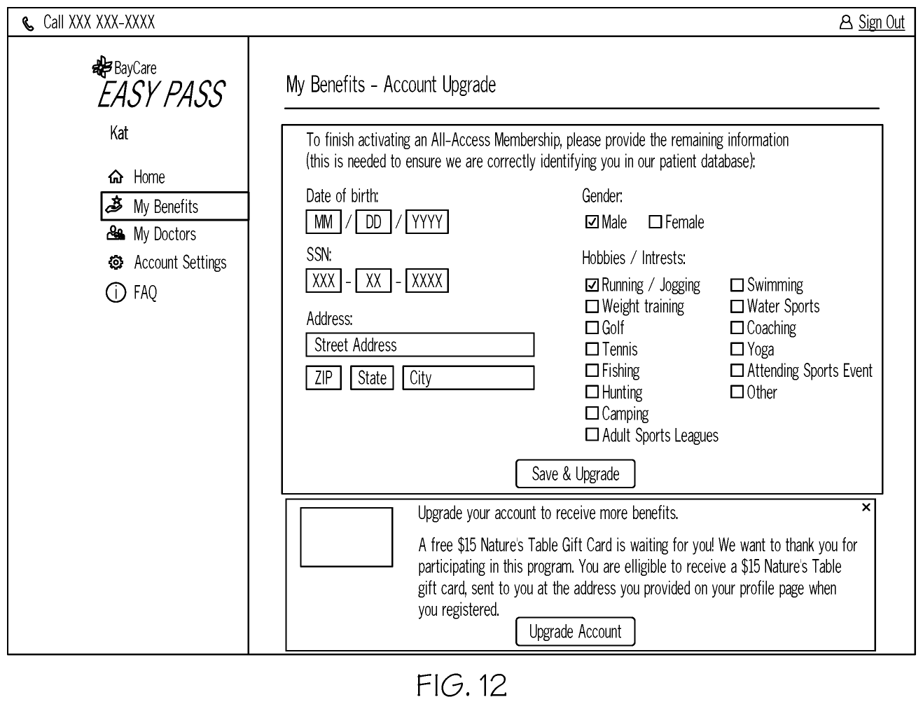

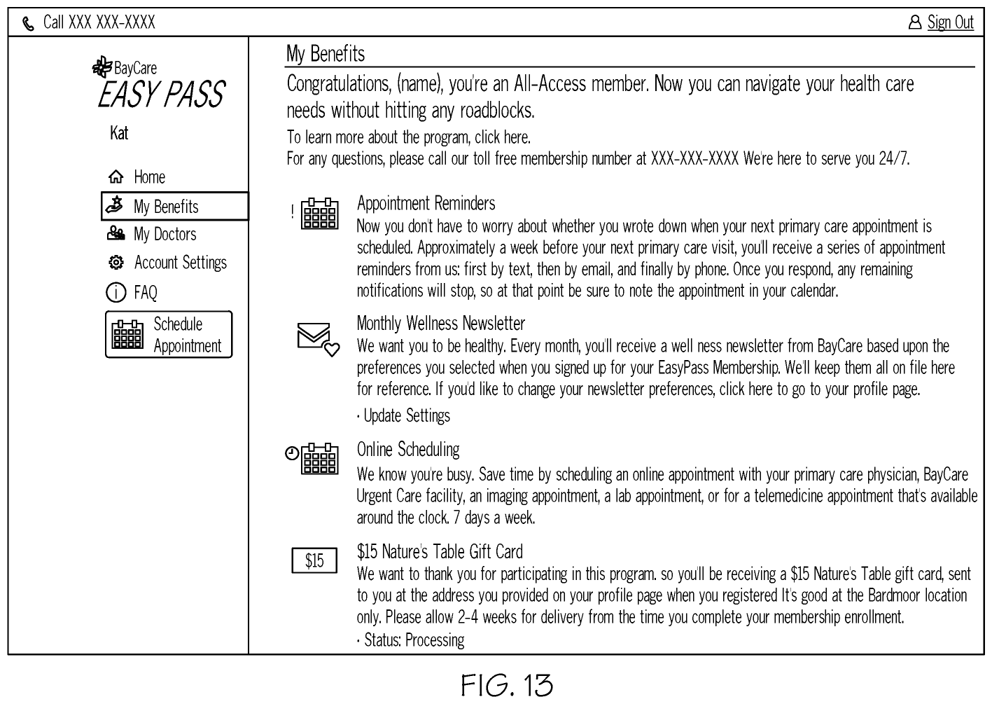

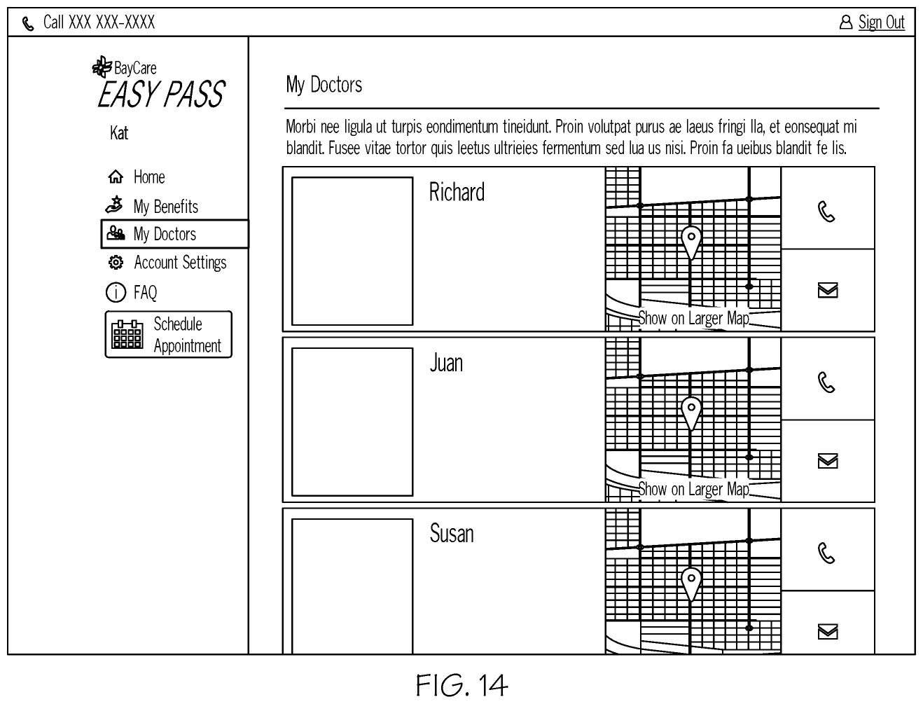

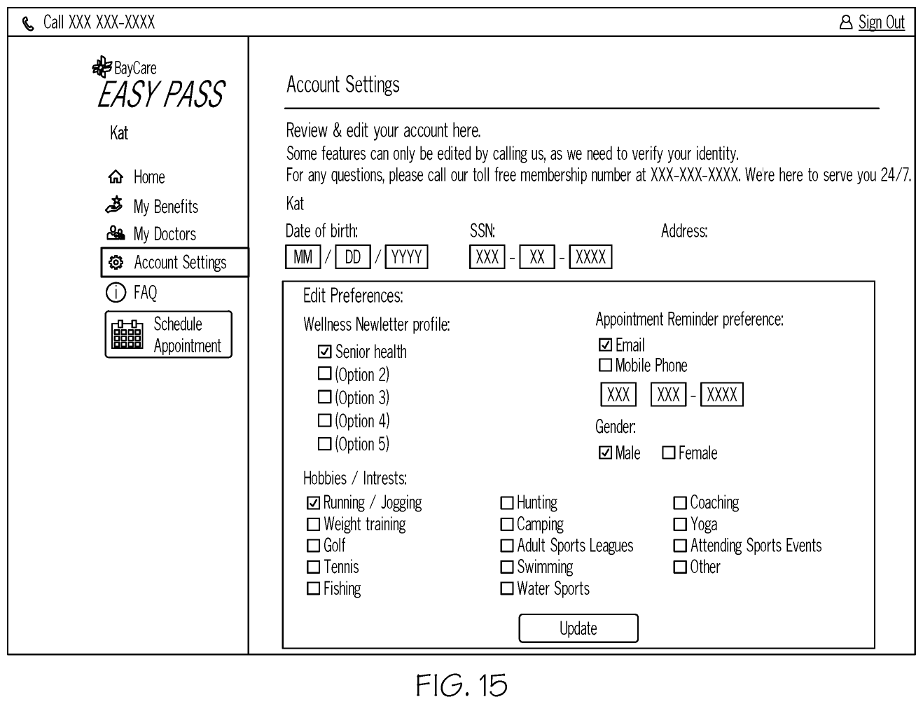

As shown in FIGS. 1-15, the invention provides systems and methods for a healthcare loyalty program. More particularly, the invention relates to systems and methods for providing gift cards to patients, who are customers of a particular healthcare provider or facility (e.g., a medical office, hospital, lab, or any other location that provides healthcare services), to promote patient health by encouraging a patient to schedule an appointment with a healthcare provider (also referred to herein as a service provider) and to build loyalty to the healthcare provider or facility and its services. The system includes a computing device that includes a processor and associated memory, a telecommunications network, a registration process, a patient database, a user validation process, a scheduling process, and a gift card process. The computing device is a mobile phone, a computer, or any other computing device having a connected or integrated display screen and inputting means (e.g., a touchscreen or physical keyboard). The system can also include other computing devices, e.g., other computers, mobile devices, or servers. For example, databases or certain processes of the system may be installed on one or more remote servers to which the computing device is communicatively connected via the telecommunications network. The telecommunications network can be the Internet, a local area network (LAN), or any other telecommunications network.

A user (typically, a patient) accesses the system via a downloadable software application or via a web browser. The downloadable software application or the non-downloadable software-as-a-service (SaaS), by which the user accesses the system, can be provided as a platform, e.g., a platform-as-a-service (PaaS). The downloadable software application or web browser are installed on the computing device, which is connected to the telecommunications network. The user accesses a registration process using the computing device. The registration process is software that is installed on the computing device or on a second computing device, e.g., on a remote server. The registration process includes a user interface that is accessible on the computing device through the downloadable software application or web browser, and allows the user to create a new account by providing user information related to the user (e.g., name, address, e-mail address, Social Security Number, date of birth, sex, or other personally identifying or user-specific user information). User information can also include information concerning the user's hobbies and interests. The user may be prompted to create a username and password. The user information entered into the registration process through the user interface of the registration process is transmitted to and stored in a database, and the user account is created.

Using the system's user validation process, the system confirms (or validates) that the user is a patient/customer of the service provider. For example, after the user submits his or her user information through the system's registration process, the registration process may transmit (at least part of) that user information to the system's user validation process. Then, the user validation process compares the user information to patient information stored in a patient database to validate whether the user is an existing patient of the service provider. If the user validation process confirms that the user is an existing patient (i.e., a validated user), the gift card process (described below) delivers a gift card to the user, e.g., before or after the user schedules an appointment with the service provider using the system's scheduling process. The system may also permit a returning user (i.e., one who already has a user account but logs in again in instances after the initial account creation) to receive a gift card by scheduling an appointment. If the user validation process confirms that the user is not an existing patient of the service provider, the system permits the user to access information (e.g., concerning healthcare topics) in the system and to schedule an appointment for a service so that the user may become a patient of the service provider.

In some embodiments, the system may permit a user to receive a gift card simply by providing information related to the user to the system even when an appointment is not scheduled.

After the user account is created, an account user interface permits the user to access a scheduling process of the system. The scheduling process presents options for one or more types of healthcare services and permits the user to schedule a date and time for such service. The system incentivizes the validated user to schedule an appointment for one or more of the healthcare services by displaying an offer in the user interface for a gift card or other reward that will be given to the user if the user schedules the appointment for the service through the system's scheduling process. Once the user enters user-selected scheduling information using the scheduling process, the scheduling process sends a command to a gift card process to generate and deliver a gift card (or other reward such as, for example, a coupon, discount card, gift certificate, credit for purchase of goods or services, or any other suitable type of reward that is a physical or electronic item or service of value) to the user. The command contains data related to the amount of the gift card, redemption information (e.g., a bar code or alphanumeric code), and in some cases, information related to the user, the user information, and/or the user account. In addition to or in lieu of a bar code or alphanumeric code, the gift card may also include a magnetic stripe, an embedded microchip, a radio-frequency identification (RFID) chip, or two or more of the foregoing in embodiments in which a physical gift card is delivered.

In some embodiments, the gift card process generates the gift card electronically and delivers it in digital form to the validated user for access within the downloadable software application, or in other embodiments, by logging into the user's account and accessing the system via the web browser. The gift card may be displayed on the display (e.g., on a screen of a mobile phone) for redemption at a retail store (including, for example, at an online retail store) by reading the redemption information. For example, reading the redemption information may be accomplished by scanning a bar code or by entry of an alphanumeric code into a retailer's point-of-sale system. In other embodiments, the redemption may be read by, for example, scanning, swiping, detecting, or otherwise reading a magnetic stripe, an embedded microchip, or a RFID chip.

In still other embodiments, the gift card process may send an instruction to a printer so that the gift card is printed and then delivered by mail, hand delivery, or other delivery means to the user.

Use of the system by patients encourages patients to schedule appointments for healthcare services with a healthcare provider because each patient receives a reward after registering an account with the system. Use of the system also builds loyalty among patients who receive rewards (i.e., the gift cards) when they schedule appointments with the service provider using the system. In some embodiments, a gift card may be delivered to the user for reasons other than scheduling an appointment such as, for example, if the user provides certain additional user information, meets a certain health objective, or reaches a predetermined spending level with the provider.

In exemplary embodiments, the system includes an electronic gift card generator. The electronic gift card generator can produce an electronic gift card that is deliverable to the user via e-mail, text message, social media, or other electronic message service. However, in exemplary embodiments, the electronic gift card is delivered to the user through a user interface of the system when the user logs into his or her account with the system. The electronic gift card may be redeemable electronically, e.g., using an e-commerce website to make a purchase of goods or services. In other embodiments, the electronic gift card may be printed by the user using a printer (e.g., a home printer or office printer) to create a physical gift card, which the user redeems in a brick-and-mortar store to purchase goods or services. The electronic gift card generator may include a communicative interface with a financial system (e.g., a bank, a credit card company, or other financial services company) that allows for funding of the gift card and processing of purchase and sale transactions using the gift card. The electronic gift card generator may also include a communicative interface with a third party gift card system that allows for issuance of the electronic gift card by a third party with whom an operator of the system has an agreement. For example, the system operator may have an agreement with a restaurant, a clothing store, another retailer of goods, another healthcare provider, or a provider of non-healthcare services to issue gift cards to the provider's customers (i.e., to the user and to other users of the system) according to rules established by the system and between the provider and the third party or between the system operator and the third party. The communicative interfaces between the system and the financial system and between the system and the third party gift card system are electronic and may include one or more financial transaction interfaces accessible by the system and by the third parties providing the gift cards or financial services. These communicative interfaces may be installed on one or more remote servers and may be accessible by the system via the telecommunications network using a computing device to which the system operator has access. The system operator may be the provider or another party.

In other embodiments, in addition to or in lieu of the electronic gift card generator, the system can include a printer capable of printing physical gift cards. The printer can receive the instruction from the gift card process, and upon receiving such instruction, can print a physical gift card for delivery to the user via any delivery means, e.g., mail, courier or shipping service, hand delivery, etc.

Two exemplary embodiments of the system are described as follows. Other embodiments in addition to those described below are also contemplated by the inventors.

### Embodiment 1 of the System

In one exemplary embodiment, the invention features a system for incentivizing a user to schedule a service with a provider by providing a reward for making an appointment. Although in this exemplary embodiment, the service is a healthcare service and the provider is a healthcare provider, in other embodiments, the service may be any other type of service including services unrelated to healthcare and the provider may be any other type of service provider including providers of services unrelated to healthcare. The system also promotes the good health of the user by increasing the likelihood of that the user will schedule an appointment for healthcare with a healthcare provider by incentivizing the user with an offer for the reward for registering with the system, making an appointment, or both. The system includes a computing device having a processor and associated memory, a display screen for displaying information and images produced or received by the computing device, and a telecommunications network. The display screen can be a computer monitor or television connected to a computer, a smart television, or a display screen of a mobile device.

The system also includes a registration process for allowing a user to create a user account by submitting user information. The registration process is accessible by the user via the telecommunications network using the computing device. The system further includes a database in which customer information related to existing customers of a provider is stored for access and retrieval, a user validation process for confirming that the user is an existing customer of the provider by comparison of the user information with customer information stored in the database, a scheduling process for the user to schedule an appointment for a service provided by the provider, and a gift card process for generating an instruction to generate and deliver a gift card to the user based on the command received from the system. The gift card is (or can include) a physical gift card, an electronic gift card, or both.

In an exemplary embodiment, the system sends a command to generate and deliver a gift card after registration is completed and the user account is created. In another embodiment, the system the system sends the command to generate and deliver the gift card after both registration is completed and an appointment has been scheduled by the user with a provider using the system. The command can be generated by the registration process or by the scheduling process. The command is transmitted to the gift card process.

The command can be or can include data related to the amount of the gift card, redemption information, or both. The redemption information of the gift card can be or can include a bar code, an alphanumeric code, or both. The command also can include information related to the user, the user information (as described elsewhere herein), the user account (e.g., the user's user name, account preferences, or other information input into the system by the user in association with the user's account or generated by the system in connection with the user's account), or a combination of two or more of the foregoing. In one embodiment, the command is generated and sent when the user account is created by the registration process. In another embodiment, the command is generated and sent after the user account is created by the registration process. In still another embodiment, the command is generated and sent when the user schedules the appointment with the provider using the scheduling process. In yet another embodiment, the command is generated and sent after the user schedules the appointment with the provider using the scheduling process.

The user validation process generates a validated user determination if the user is determined to be an existing customer of the provider and a non-user determination if the user is determined not to be an existing customer of the provider.

The scheduling process analyzes the customer information in the database and makes an affirmative determination if the customer information includes at least one record of an appointment being scheduled and completed by the user and makes a negative determination if the customer information does not include at least one record of an appointment being scheduled and completed by the user. In one embodiment, the scheduling process sends the command to the gift card process only if the appointment is a first appointment by the user with the provider. In other embodiments, the scheduling process sends the command to the gift card process only after an appointment that is subsequent to the user's first appointment, for example, after a second appointment, a third appointment, a fourth appointment, a fifth appointment, a tenth appointment, a twentieth appointment, etc. In still other embodiments, commands are sent after more than one appointment, e.g., after the first appointment and after the second appointment, after the first appointment and after the third appointment, etc., so that the user receives a reward (e.g., a gift card) after each such appointment. In some embodiments, the user receives a reward only after an appointment subsequent to the first appointment. In other embodiments, the user receives a reward only after the first appointment. In still other embodiments, the user receives a reward after each appointment. In yet other embodiments, the user receives a reward after only certain appointments based on criteria established by an operator of the system (e.g., after so many appointments have been scheduled and attended, after a certain amount of spending by the user, after certain services are provided to the user, etc.). In other embodiments, the user receives a reward after providing information related to the user to the system even if no appointment is scheduled or even if an appointment is scheduled but not attended.

The scheduling process accesses a database (i.e., a scheduling database) to determine if the appointment is a first appointment with the provider or a subsequent appointment. This database includes records of each appointment scheduled and attended by the user. This database can be the same database in which customer information related to existing customers of a provider is stored for access and retrieval (i.e., the user information database), or it can be a different database. In embodiments in which the scheduling database and the user information database are two separate databases, each database may be stored on a different computing device, for example, on two separate remote servers.

The records of each appointment, which are stored in the scheduling database, can include one or more scheduled appointment records and one or more attended appointment records that correspond to the one or more scheduled appointment records. Each of the one or more scheduled appointment records has a corresponding attended appointment record. Each of the one or more attended appointment records includes an “attended” record if the user attended the scheduled appointment and a “not attended” record if the user did not attend the scheduled appointment. In embodiments of the system in which the command is sent to gift card process after an appointment is scheduled or attended, the scheduled appointment records and attended appointment records may be analyzed by the scheduling process so that the scheduling process may make a determination to send the command to the gift card process or not.

As mentioned above, the gift card (or other reward) may be sent to the user only after an appointment has been scheduled, or in other embodiments, only after the user has attended a scheduled appointment. In some embodiments, the scheduling process may send the command to the gift card process only if the appointment is a first appointment by the user with the provider. In other embodiments, the scheduling process may send the command to the gift card process if the appointment is a first appointment or a subsequent appointment by the user with the provider.

### Embodiment 2 of the System

In another exemplary embodiment, the invention features a system for incentivizing a user to schedule a service with a provider by providing a reward for making an appointment. In this embodiment also, the system also promotes the good health of the user by increasing the likelihood of that the user will schedule an appointment for healthcare with a healthcare provider by incentivizing the user with an offer for the reward for registering with the system, making an appointment, or both. The system includes a computing device having a processor and associated memory, a display screen for displaying information and images produced or received by the computing device, and a telecommunications network.

The system also includes a registration process for allowing a user to create a user account by submitting user information. The registration process is accessible by the user via the telecommunications network using the computing device. The system includes a database in which customer information related to existing customers of a provider is stored for access and retrieval via the telecommunications network using the computing device. The system also includes a user validation process for confirming that the user is an existing customer of the provider by comparison of user information associated with the user with customer information stored in the database. The system further includes a scheduling process for the user to schedule an appointment for a service provided by the provider. The system also includes a reward process for generating an instruction to generate and deliver a reward to the user based on the command received from the registration process or the scheduling process. The reward is or includes a physical or electronic item of value. In some embodiments, the reward can be or can include a gift card. The gift card can be or include a physical gift card, an electronic gift card, or both.

Methods

The invention also relates to a method that can be used for incentivizing a user to schedule a service with a provider by providing a reward for making an appointment. The method includes the step of providing a system that includes a computing device having a processor and associated memory, a display screen for displaying information and images produced or received by the computing device, and a telecommunications network. The system of the method also includes a registration process for allowing a user to create a user account by submitting user information. The registration process is accessible by the user via the telecommunications network using the computing device. The system of the method also includes a database in which customer information related to existing customers of a provider is stored for access and retrieval via the telecommunications network using the computing device. The system of the method further includes a user validation process for confirming that the user is an existing customer of the provider by comparison of user information associated with the user with customer information stored in the database. The system of the method also includes a scheduling process for the user to schedule an appointment for a service provided by the provider. The system of the method further includes a reward process for generating an instruction to generate and deliver a reward to the user based on the command received from the registration process or the scheduling process. The system of the method may also include any other components, parts, equipment, processes, and other features as are described elsewhere herein as being included in various embodiments of the system.

The method also includes the step of creating the user's user account using the registration process to submit the user's user information. The method also includes the step of confirming that a user is an existing customer of the provider by using the user validation process to compare user information associated with the user with customer information stored in the database. The method also includes the step of scheduling an appointment for the user using the scheduling process for a service provided by the provider. The method also includes the step of sending a command to the reward process to generate and deliver a reward. The method also includes the step of generating an instruction via the reward process to generate and deliver a reward to the user based on the command. The reward is (or can include) a physical or electronic item of value or both.

In the method, the reward can be or can include a gift card. The gift card can be or can include a physical gift card, an electronic gift card, or both.

In some embodiments, the method also can include the step of analyzing the customer information in the database and making an affirmative determination if the customer information includes at least one record of an appointment being scheduled and completed by the user and making a negative determination if the customer information does not include at least one record of an appointment being scheduled and completed by the user.

In some embodiments, the method also can include the step of sending the command to the reward process only if the appointment is a first appointment by the user with the provider. In other embodiments, the method can include the step of sending the command to the reward process if the appointment is a first appointment or a subsequent appointment by the user with the provider. In still other embodiments, the method can include the step of sending the command to the reward process only if the appointment is a subsequent appointment by the user with the provider. In some embodiments, the user receives a reward only after an appointment subsequent to the first appointment. In other embodiments, the user receives a reward only after the first appointment. In still other embodiments, the user receives a reward after each appointment. In yet other embodiments, the user receives a reward after only certain appointments based on criteria established by an operator of the system (e.g., after so many appointments have been scheduled and attended, after a certain amount of spending by the user, after certain services are provided to the user, etc.). In other embodiments, the user receives a reward after providing information related to the user to the system even if no appointment is scheduled or even if an appointment is scheduled but not attended.

In some embodiments, the method also can include the step of accessing a database using the scheduling process and determining if the appointment is a first appointment with the provider or a subsequent appointment, wherein the database includes records of each appointment scheduled and attended by the user. The records of each appointment can include one or more scheduled appointment records and one or more attended appointment records that correspond to the one or more scheduled appointment records. Each of the one or more scheduled appointment records includes a corresponding attended appointment record. Each of the one or more attended appointment records includes an “attended” record if the user attended the scheduled appointment and a “not attended” record if the user did not attend the scheduled appointment.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A system for incentivizing a user to schedule a service with a provider by providing a reward after accessing a platform for making an appointment, the system comprising:
a computing device comprising a processor and associated memory;
a display screen for displaying information and images produced or received by the computing device;
a telecommunications network;
a registration process for allowing a user to create a user account by submitting user information, wherein the registration process is accessible by the user via the telecommunications network using the computing device;
a database in which customer information related to existing customers of a provider is stored for access and retrieval;
a user validation process for confirming that the user is an existing customer of the provider by comparison of the user information with customer information stored in the database;
a scheduling process for the user to schedule an appointment for a service provided by the provider; and
a gift card process for generating an instruction to generate and deliver a gift card to the user based on a command received from the system;
wherein the system sends the command to generate and deliver the gift card after registration is completed and the user account is created.

2. The system of claim 1, wherein the command is generated by the registration process or by the scheduling process.

3. The system of claim 1, wherein the command comprises data related to the amount of the gift card, redemption information, or both.

4. The system of claim 2, wherein the redemption information of the gift card comprises a bar code, an alphanumeric code, or both.

5. The system of claim 2, wherein the command further comprises information related to the user, the user information, the user account, or a combination of two or more of the foregoing.

6. The system of claim 1, wherein the user validation process generates a validated user determination if the user is determined to be an existing customer and a non-user determination if the user is determined not to be an existing customer.

7. The system of claim 1, wherein the command is generated and sent when or after the user account is created by the registration process.

8. The system of claim 1, wherein the command is generated and sent when or after the user schedules the appointment with the provider using the scheduling process.

9. The system of claim 1, wherein the gift card comprises a physical gift card, an electronic gift card, or both.

10. The system of claim 1, wherein the scheduling process analyzes the customer information in the database and makes an affirmative determination if the customer information comprises at least one record of an appointment being scheduled and completed by the user and makes a negative determination if the customer information does not comprise at least one record of an appointment being scheduled and completed by the user.

11. The system of claim 1, wherein the scheduling process sends the command to the gift card process only if the appointment is a first appointment by the user with the provider.

12. The system of claim 1, wherein the scheduling process accesses a database to determine if the appointment is a first appointment with the provider or a subsequent appointment, wherein the database comprises records of each appointment scheduled and attended by the user.

13. The system of claim 12, wherein the records of each appointment comprise one or more scheduled appointment records and one or more attended appointment records that correspond to the one or more scheduled appointment records, wherein each of the one or more scheduled appointment records has a corresponding attended appointment record, wherein each of the one or more attended appointment records comprises an “attended” record if the user attended the scheduled appointment and a “not attended” record if the user did not attend the scheduled appointment.

14. The system of claim 12, wherein the scheduling process sends the command to the gift card process only if the appointment is a first appointment by the user with the provider.

15. The system of claim 12, wherein the scheduling process sends the command to the gift card process if the appointment is a first appointment or a subsequent appointment by the user with the provider.

16. A system for incentivizing a user to schedule a service with a provider by providing a reward after accessing a platform for making an appointment, the system comprising:
a computing device comprising a processor and associated memory;
a display screen for displaying information and images produced or received by the computing device;
a telecommunications network;
a registration process for allowing a user to create a user account by submitting user information, wherein the registration process is accessible by the user via the telecommunications network using the computing device;
a database in which customer information related to existing customers of a provider is stored for access and retrieval via the telecommunications network using the computing device;
a user validation process for confirming that the user is an existing customer of the provider by comparison of user information associated with the user with customer information stored in the database;
a scheduling process for the user to schedule an appointment for a service provided by the provider; and
a reward process for generating an instruction to generate and deliver a reward to the user based on a command received from the registration process or the scheduling process, wherein the reward comprises a physical or electronic item of value.

17. The system of claim 16, wherein the reward comprises a gift card; and wherein the gift card comprises a physical gift card, an electronic gift card, or both.

18. A method for incentivizing a user to schedule a service with a provider by providing a reward after accessing a platform for making an appointment, the method comprising the steps of:
(a) providing a system comprising:
(i) a computing device comprising a processor and associated memory;
(ii) a display screen for displaying information and images produced or received by the computing device;
(iii) a telecommunications network;
(iv) a registration process for allowing a user to create a user account by submitting user information, wherein the registration process is accessible by the user via the telecommunications network using the computing device;
(v) a database in which customer information related to existing customers of a provider is stored for access and retrieval via the telecommunications network using the computing device;
(vi) a user validation process for confirming that the user is an existing customer of the provider by comparison of user information associated with the user with customer information stored in the database;
(vii) a scheduling process for the user to schedule an appointment for a service provided by the provider; and
(viii) a reward process for generating an instruction to generate and deliver a reward to the user based on a command received from the registration process or the scheduling process;
(b) creating the user's user account using the registration process to submit the user's user information;
(c) confirming that a user is an existing customer of the provider by using the user validation process to compare user information associated with the user with customer information stored in the database;
(d) scheduling an appointment for the user using the scheduling process for a service provided by the provider;
(e) sending a command to the reward process to generate and deliver a reward; and
(f) generating an instruction via the reward process to generate and deliver a reward to the user based on the command, wherein the reward comprises a physical or electronic item of value.

19. The method of claim 18, wherein the reward comprises a gift card; and wherein the gift card comprises a physical gift card, an electronic gift card, or both.

20. The method of claim 18, further comprising the step of:
(g) analyzing the customer information in the database and making an affirmative determination if the customer information comprises at least one record of an appointment being scheduled and completed by the user and making a negative determination if the customer information does not comprise at least one record of an appointment being scheduled and completed by the user.

21. The method of claim 18, further comprising a step selected from the group consisting of:
(h) sending the command to the reward process only if the appointment is a first appointment by the user with the provider;
(i) sending the command to the reward process if the appointment is a first appointment or a subsequent appointment by the user with the provider; and
(j) sending the command to the reward process only if the appointment is a subsequent appointment by the user with the provider.

22. The method of claim 18, further comprising the step of:
k) accessing a database using the scheduling process and determining if the appointment is a first appointment with the provider or a subsequent appointment, wherein the database comprises records of each appointment scheduled and attended by the user.

23. The method of claim 18, wherein the records of each appointment comprise one or more scheduled appointment records and one or more attended appointment records that correspond to the one or more scheduled appointment records, wherein each of the one or more scheduled appointment records comprises a corresponding attended appointment record, wherein each of the one or more attended appointment records comprises an “attended” record if the user attended the scheduled appointment and a “not attended” record if the user did not attend the scheduled appointment.
